SEQUENCE LISTING (3 sequences)

SEQ ID NO: 1 - protein, 41 aa, Homo sapiens
MGPRRLLLVAACFSLCGPLLSARTRARRPESKATNATLDPR

SEQ ID NO: 2 - protein, 13 aa, Homo sapiens
SARTRARRPESKA

SEQ ID NO: 3 - protein, 20 aa, Homo sapiens
RRLLLVAACFSLCGPLLSAR

(19) United States
(12) Patent Application Publication
(10) Pub. No.: US 2012/0088311 A1
(43) Pub. Date: Apr. 12, 2012

(54) Immuno-Detection of a Cancerous State in a Subject

(75) Inventor: Rachel Bar-Shavit, Jerusalem (IL)

(73) Assignee: HADASIT MEDICAL RESEARCH SERVICES & DEVELOPMENT LIMITED, JERUSALEM (IL)

(21) Appl. No.: 12/866,683

(22) PCT Filed: Feb. 5, 2009
(86) PCT No.: PCT/IL09/00135

§ 371 Date: Aug. 6, 2010

Related U.S. Application Data
(60) Provisional application No. 61/006,949, filed on Feb. 7, 2008.

(51) Int. Cl. G01N 33/574

(52) U.S. Cl. 436/501

(57) ABSTRACT

The present invention is based on the finding that antibodies raised against a fragment of PAR1-released peptide may be used to detect in a bodily fluid sample from a subject a marker associated with cancer state, if said subject has cancer. Thus, the present invention provides the methods and packages for conducting one or more of the following: determining a cancerous state in a subject, the method comprises determining binding of an antibody raised against a protease-activated receptor 1 (PAR1) released peptide or a fragment derived therefrom to a marker within a fluid sample obtained from said subject, wherein binding of said antibody to said marker being indicative of a cancerous state; determining severity of a cancerous state in a subject comprising determining level of binding of an antibody raised against PAR1 released peptide or a fragment derived therefrom to a marker within a fluid sample obtained from said subject, and comparing the level of binding with the level of prior determined standards that correlate level of antibody binding to PAR1 released peptide with severity of cancerous state; and determining the effectiveness of a therapeutic treatment of a subject with an anti-cancer agent to the subject comprising determining the level of binding of an antibody raised against PAR1 released peptide or a fragment derived therefrom to a marker within a fluid sample obtained from said subject in two or more successive time points, one or more time points are during the therapeutic treatment, wherein a difference in the level being indicative of effectiveness of therapeutic treatment.

### FIELD OF THE INVENTION

This invention relates to diagnosis of cancer and more particularly to immuno-detection of a cancerous state in a subject.

### PRIOR ART

The following is a list of art which is considered to be pertinent for describing the state of the art in the field of the invention. Acknowledgement of these references herein will at times be made by indicating their number within brackets from the list below.

Claytor R B et al., Journal of Vascular Surgery, Volume 37, Issue 2, Pages 440-445

Furman M I, Liu L, Benoit S E, Becker R C, Barnard M R, Michelson A D. The cleaved peptide of the thrombin receptor is a strong platelet agonist. Proc Natl Acad Sci USA. 1998 Mar 17; 95(6):3082-7

Coughlin S R. How the protease thrombin talks to cells. Proc Natl Acad Sci USA. 1999 Sep. 28; 96(20):11023-7

Lerner D J, Chen M, Tram T, Coughlin S R. Agonist recognition by proteinase-activated receptor 2 and thrombin receptor. Importance of extracellular loop interactions for receptor function. J Biol Chem. 1996 Jun. 14; 271(24):13943-7

Hammes S R, Shapiro M J, Coughlin S R. Shutoff and agonist-triggered internalization of protease-activated receptor 1 can be separated by mutation of putative phosphorylation sites in the cytoplasmic tail. Biochemistry. 1999 Jul. 20; 38(29):9308-16

Golz S, Brueggemeier U L F and Summer H. International Patent Application Publication number WO 2004/081044

Hoxie J A, Brass L. International Patent Application Publication number WO 2007/46879

### BACKGROUND OF THE INVENTION

Protease Activated Receptors (PARs) are seven transmembrane G-coupled receptors (GPCR) that are uniquely activated by proteolytic cleavage. Four different PARs have been identified (PAR1-4), all responding to a highly select group of serine proteases. The PARs act as sensitive sensors of extra cellular protease gradients to allow cells to respond to proteolytically modified environment. While traditionally PAR1 plays a role in thrombosis, hemostasis and vascular biology, it emerges with surprisingly new assignment in tumor biology. This is supported by the pattern of PAR1 expression in normal and pathological epithelia. In addition, a cDNA expression library screen based on the loss of anchorage—dependent growth and focus forming activity in NIH3T3 cells led to the isolation of PAR1 as a novel oncogene. Thus, PAR1 joins a list of GPCRs that are oncogenes including mas and g2a. The oncogenic properties of PAR1, along with ample evidence on the high expression levels of the human Par1 (hPar1) gene in tumor biopsy specimens and in differentially metastatic cell lines—point to a direct correlation between PAR1 expression and the degree of malignancy. PAR1 has been shown to be involved in a variety of primary human cancers including those of breast (Even-Ram S, et al. (1998) Nat Med. 4(8):909-14.); colon (Vergnolle N, et al. (2004) J Clin Invest.114 (10):1444-56; Darmoul D, et al. (2004) Mol Cancer Res. 2(9):514-22.); prostate (Chay C H, et al. Urology (2002) 60(5):760-5; Salah Z, et al. (2005) FASEB J 19(1):62-72); ovary (Grisaru-Granovsky S, et al. 2005. Differential expression of protease activated receptor 1 (Par1) and pY397FAK in benign and malignant human ovarian tissue samples. Int J Cancer 113(3):372-8); and melanoma (Nierodzik M L, et al. (1998) Blood. 92(10):3694-700; Shi X,et al. (2004) Mol Cancer Res. 2(7):395-402).

The fact that PAR1 gene and protein over expression are associated with the aggressiveness of tumors in vivo, reflect on its potential role in tumor dissemination and assigns it as an attractive target for anticancer therapy. In-fact, PAR1 plays a central role at least, in breast tumor progression since introduction of an hPar1 antisense sequence (a plasmid of 462 base pairs antisense sequence containing part of the promoter and the start initiation site of the protein) reduces their ability to migrate through Matrigel coated filters, in vitro (Even-Ram S, et. al.(1998) Nat Med. 4(8):909-14).

Recently, a 41 residue polypeptide released from PAR1 (TR1-41) has been shown to be a strong platelet agonist (Claytor R B et al., Journal of Vascular Surgery, Volume 37, Issue 2, Pages 440-445 R).

Some additional publications relating to PAR1 include:

WO 2007/020645 describing nucleic acid molecules, vectors, compositions, and methods useful for modulating protease-activated receptor 1 gene expression via RNA interference. In particular, the publication features small interfering RNA (siRNA) and short hairpin RNA (shRNA) molecules and methods for modulating the expression of protease-activated receptor 1 gene.

WO 2004/081044 describing a human PAR1 associated with the cardiovascular disorders, dermatological disorders, gastrointestinal and liver diseases, neurological disorders, cancer disorders and urological disorders; to assays for the identification of compounds useful in the treatment or prevention of these disorders and diseases and to compounds which bind to and/or activate or inhibit the activity of PAR1 as well as pharmaceutical compositions comprising such compounds.

WO 2007/46879 describing methods and kits for detecting thrombin-induced cell activation via a system of detection capable of determining the presence of the cleaved peptide fragment of the thrombin receptor.

### SUMMARY OF THE INVENTION

The present invention provides, in accordance with one aspect, a method of determining a cancerous state in a subject, the method comprises determining binding of an antibody raised against a protease-activated receptor 1 (PAR1) released peptide or a fragment derived therefrom to a marker within a fluid sample obtained from said subject, wherein binding of said antibody to said marker being indicative of a cancerous state.

In accordance with another aspect, the invention provides, a method for determining severity of a cancerous state in a subject comprising determining level of binding of an antibody raised against PAR1 released peptide or a fragment derived therefrom to a marker within a fluid sample obtained from said subject, and comparing the level of binding with the level of prior determined standards that correlate level of antibody binding to PAR1 released peptide with severity of cancerous state.

In accordance with yet another aspect, the present invention provides a method for determining the effectiveness of a therapeutic treatment of a subject with an anti-cancer agent to the subject comprising determining the level of binding of an antibody raised against PAR1 released peptide or a fragment derived therefrom to a marker within a fluid sample obtained from said subject in two or more successive time points, one or more time points are during the therapeutic treatment, wherein a difference in the level being indicative of effectiveness of therapeutic treatment.

In one embodiment, the PAR1 released peptide comprises or consists of the sequence depicted as SEQ ID NO:1. The fragment of said PAR1-released peptide, in accordance with one embodiment comprises or consists of the sequence identified herein as SEQ ID NO:2 or SEQ ID NO:3, preferably SEQ ID NO:3.

The invention also provides, in accordance with another aspect, a package for determining binding of an antibody raised against a protease-activated receptor 1 (PAR1) released peptide or a fragment thereof to a marker within a fluid sample obtained from a subject, comprising:

at least one antibody raised against a protease-activated receptor 1 (PAR1) released peptide or a fragment thereof and capable of binding to said marker if present in said fluid sample;

(ii) instructions for use of said at least one antibody for determining one or more of:
binding of said antibody to said marker;
level of binding of said antibody to said marker;
difference between level of binding of said antibody to said marker and level of prior determined standards that correlate level of antibody binding to PAR1 released peptide with severity of cancerous state;
difference in level of binding of at least one antibody to said marker in two or more successive fluid samples from the same subject.

### BRIEF DESCRIPTION OF THE DRAWINGS

In order to understand the invention and to see how it may be carried out in practice, embodiments will now be described, by way of non-limiting example only, with reference to the accompanying drawings, in which:

FIGS. 1A-1B show the binding of polyclonal antibodies to different amounts (5, 10, 20, 30 ng) of a peptide derived from PAR1-released peptide, namely, peptide PAR1rp2 obtained by western blot analysis (FIG. 1A); the results being presented also as a function of the relative band intensity (FIG. 1B).

FIG. 2 is a western blot analysis showing the binding of polyclonal antibodies to different amounts (20, 50 and 100 ng) of the 41 amino acid containing PAR1-released peptide (appearing on the gel as 35 kDa in size) and to the peptide derived therefore, PAR1rp2 spiked into serum of healthy subjects (appearing on the gel as 6 kDa in size), thereby demonstrating that the polyclonal antibodies raised against PAR1rp2 recognize the larger, PAR1-released peptide or a fragment thereof in complex with another serum component.

FIGS. 3A-3B are western blot analyses of the binding of polyclonal antibodies to a marker in serum samples; FIG. 3A illustrates serum samples from healthy subjects (H-n, n being an arbitrary integer representing different subjects) and a control sample of a healthy sample spiked with PAR1 derived synthetic peptide, PAR1rp2 (H-8+synthetic peptide) and FIG. 3B illustrates serum samples from cancer patients (C-n, n being an arbitrary integer representing different cancer patients) vs. a control sample of a healthy subject a control sample spiked with the PAR1 derived synthetic peptide, PAR1rp2 (+H).

FIG. 4A-4C are western blot analyses of binding of polyclonal antibodies to a marker in serum samples from stage IV cancer patients (C-i or C-ii, i and ii representing two different cancer patients) before (FIG. 4A) and after receiving anti-cancer treatment (FIG. 4B), and a control sample of serum from a healthy subject spiked with PAR1 derived synthetic peptide, PAR1rp2 (+H) (FIG. 4C).

FIGS. 5A-5C are western blot analyses of binding of polyclonal antibodies to a marker in serum samples from cancer patients (Cx, and Cy, representing two different cancer patients) (FIG. 5A), or from a trauma subject (FIG. 5B) at the day of trauma (day of injury, (T)), or 3 days (T+3D) or 6 days (T+6D) post trauma and a control serum sample from a healthy subject spiked with PAR1 derived synthetic peptide, PAR1rp2 (+H) (FIG. 5C).

FIG. 6 is a western blot analysis of binding of polyclonal antibodies to a marker in serum samples from cancer patients (C-10, C-11, C-12, C-13, C-14 and C-15) at different stages of the disease, and control serum samples from a healthy subject (H-10) spiked with PAR1 derived synthetic peptide, PAR1rp2 (H-10+peptide) or without peptide (H-10).

### DETAILED DESCRIPTION OF SOME NON-LIMITING EMBODIMENTS OF THE INVENTION

The present invention is based on the surprising finding that polyclonal antibodies raised against a short amino acid peptide (herein referred to as“PAR1rp2”), derived from protease-activated receptor 1 (PAR 1)-released peptide, was capable of binding to a component of blood samples obtained from breast cancer patients, while no such binding was detected with respect to blood samples obtained from individuals which did not have breast cancer (healthy subjects). Thus, it was envisages that the detection of PAR1-released peptide within a bodily fluid sample, such as a blood sample, is reflecting (mirroring) a cancerous state of a tested subject and thus it was suggested herein to be a diagnostic tool for determining the cancerous state.

Thus, based on these finding there is disclosed herein a method of determining a cancerous state in a subject, the method comprises determining binding of an antibody raised against PAR1-released peptide or a fragment derived therefrom to a marker within a fluid sample obtained from said subject, wherein binding of said antibody to said marker being indicative of said cancerous state.

The present invention is also based on the finding that the level of binding of the antibody to the blood component can be determined and this allows determination of the severity of the cancer disease.

Thus, also disclosed herein is a method for determining severity of a cancerous state in a subject comprising determining level of binding of an antibody raised against a PAR1-released peptide or a fragment derived therefrom to a marker within a fluid sample obtained from said subject, and comparing the level of binding with the level of prior determined standards that correlate level of antibody binding to PAR1 released peptide with the severity of a cancerous state.

Further, the present invention is based on the finding that treatment of subjects having cancer with an anti-cancer agent affects the level of binding of the antibody to the blood component.

Thus, also disclosed herein is a method for determining the effectiveness of a therapeutic treatment of a subject with an anti-cancer agent comprising determining the level of binding of an antibody raised against PAR1-released peptide or fragment derived therefrom to a marker within a fluid sample obtained from said subject in two or more successive time points, one or more of which is during the therapeutic treatment, wherein a difference in the level being indicative of effectiveness of the therapeutic treatment.

PAR1-released peptide may be any peptide or polypeptide comprising a sequence of at least 5, preferably at least 6, and more preferably, at least 7, amino acid residues cleaved from PAR1 (therefore the peptide is referred to by the term “PAR1-released peptide”). It is noted that the sequence of at least 5, preferably at least 6, and more preferably, at least 7, amino acid residues may be part of a chimeric peptide, namely, the sequence being flanked by non-related sequence(s).

In one embodiment of the invention, PAR1-released peptide is a 41 amino acid residues peptide derived from the N-terminal of PAR1 and preferably consisting of the sequence:

(SEQ ID NO: 1)
NH₂-Met-Gly-Pro-Arg-Arg-Leu-Leu-Leu-Val-Ala-Ala-
Cys-Phe-Ser-Leu-Cys-Gly-Pro-Leu-Leu-Ser-Ala-Arg-
Thr-Arg-Ala-Arg-Arg-Pro-Glu-Ser-Lys-Ala-Thr-Asn-
Ala-Thr-Leu-Asp-Pro-Arg-COOH

According to the present disclosure, the antibody may be raised against a fragment derived from said PAR1-released peptide. The fragment may comprise between 5 to 41, at times, between 7-35, and even at times between 15-25 amino acids, having at least 70%, at least 80%, at least 90%, at least 95% and even at least 99% identity with the original PAR1-released peptide, when the sequence of the fragment and the sequence of the original PAR1-released peptide are optimally aligned. Typically, an amino acid sequence of at least about 5-10 is required in order to elicit the production of antibodies. Thus, in one embodiment, the antibodies are raised against a peptide comprising at least 5-10 residues having at least 70%, at least 80%, at least 90%, at least 95% and even at least 99% identity with the original PAR1-released peptide, when the sequence of the fragment and the sequence of the original PAR1-released peptide are optimally aligned.

The term “optimally aligned” is used to denote an alignment of two amino acids sequences (e.g. of the PAR1-released peptide and the fragment thereof) giving the highest percent identity score.

In one embodiment, the fragment comprises a sequence of between about 15 to 25 amino acid residues, the sequence being identical to a portion of the PAR1-released peptide when the two fragments and the peptide are optimally aligned.

In the context of the present disclosure, the fragment, being derived from PAR1-released peptide, is any amino acid sequence comprising at least 5 consecutive amino acid residues corresponding to consecutive amino acid residues in the PAR1-released peptide, when the fragment and the PAR1-released peptide are optimally aligned. In another embodiment, the fragment is any amino acid sequence comprising at least 5, 6, 7, 8, 9, or 10 consecutive amino acid residues corresponding to consecutive amino acid residues in the PAR1-released peptide, when the fragment and the PAR1-released peptide are optimally aligned.

The at least 5, 6, 7, 8, 9, or 10 consecutive amino acid residues in the fragment may be identical to the corresponding consecutive amino acid residues in said PAR1-released peptide or may comprise one or more conservative modifications of the original sequence, namely, of the naturally occurring sequence of PAR1-released peptide.

The term “conservative modification” is used to denote a modification to the “original” PAR1-released peptide including conservative replacement (substitution) of one or more naturally occurring amino acid with a non-naturally occurring amino acid, insertion or deletion of one or more amino acids as well as chemically modification of an amino acid, as appreciated by those versed in the art. The modification may also include alteration of a bond within the peptidic backbone.

The term “naturally occurring amino acid” refers to a moiety found within a peptide and is represented by —NH—CHR—CO—, wherein R corresponds to the side chain of the 20 naturally appearing amino acids. The term “non-naturally occurring amino acid” (amino acid analog) is either a peptidomimetic organic moiety, or is a D or L residue having the following formula: —NH—CHR—CO—, wherein R is an aliphatic group, a substituted aliphatic group, a benzyl group, a substituted benzyl group, an aromatic group or a substituted aromatic group and wherein R does not correspond to the side chain of a naturally-occurring amino acid. This term also refers to the D-amino acid counterpart of naturally occurring amino acids. Amino acid analogs are well known in the art; a large number of these analogs are commercially available.

The term “conservative replacement” in the context of the present invention refers to the replacement of an original amino acid residue present in the PAR1-released peptide with a naturally or non-naturally occurring amino having similar steric properties. Where the side-chain of the original amino acid residue to be replaced is either polar or hydrophobic, the conservative substitution should be with a naturally occurring amino acid or a non-naturally occurring amino acid which is also polar or hydrophobic (in addition to having the same steric properties as the side-chain of the replaced amino acid); where the original amino acid to be replaced is charged, the conservative substitution should be with a naturally occurring amino acid, or a non-naturally occurring amino acid which are charged, or the original charged amino acid may be replaced with non-charged (polar, hydrophobic) amino acids that has the same steric properties as the side-chain of the replaced amino acid.

For example in accordance with the invention the following substitutions are considered as conservative: replacement of arginine by cytroline; arginine by glutamine; aspartate by asparagine; glutamate by glutamine.

For producing conservative substitutions by non-naturally occurring amino acids it is also possible to use amino acid analogs (synthetic amino acids) well known in the art. A peptidomimetic of the naturally occurring amino acid is well documented in the literature known to the skilled practitioner. The following are some non-limiting examples of groups of naturally occurring amino acids or of amino acid analogs are listed bellow. Replacement of one member in the group by another member of the group will be considered herein as conservative substitutions:

Group I includes leucine, isoleucine, valine, methionine, phenylalanine, serine, cysteine, threonine and modified amino acids having the following side chains: ethyl, n-butyl, —CH₂CH₂OH, —CH₂CH₂CH₂OH, —CH₂CHOHCH₃ and —CH₂SCH₃. Preferably Group I includes leucine, isoleucine, valine and methionine.

Group II includes glycine, alanine, valine, serine, cysteine, threonine and a modified amino acid having an ethyl side chain. Preferably Group II includes glycine and alanine.

Group III includes phenylalanine, phenylglycine, tyrosine, tryptophan, cyclohexylmethyl, and modified amino residues having substituted benzyl or phenyl side chains. Preferred substituents include one or more of the following: halogen, methyl, ethyl, nitro, methoxy, ethoxy and —CN. Preferably, Group III includes phenylalanine, tyrosine and tryptophan.

Group IV includes glutamic acid, aspartic acid, a substituted or unsubstituted aliphatic, aromatic or benzylic ester of glutamic or aspartic acid (e.g., methyl, ethyl, n-propyl iso-propyl, cyclohexyl, benzyl or substituted benzyl), glutamine, asparagine, CO—NH-alkylated glutamine or asparagine (e.g., methyl, ethyl, n-propyl and iso-propyl) and modified amino acids having the side chain —(CH₂)₃COOH, an ester thereof (substituted or unsubstituted aliphatic, aromatic or benzylic ester), an amide thereof and a substituted or unsubstituted N-alkylated amide thereof. Preferably, Group IV includes glutamic acid, aspartic acid, glutamine, asparagine, methyl aspartate, ethyl aspartate, benzyl aspartate and methyl glutamate, ethyl glutamate and benzyl glutamate.

Group V includes histidine, lysine, arginine, N-nitroarginine, β-cycloarginine, μ-hydroxyarginine, N-amidinocitruline and 2-amino-4-guanidinobutanoic acid, homologs of lysine, homologs of arginine and ornithine. Preferably, Group V includes histidine, lysine, arginine, and ornithine. A homolog of an amino acid includes from 1 to about 3 additional methylene units in the side chain.

Group VI includes serine, threonine, cysteine and modified amino acids having C₁-C₅ straight or branched alkyl side chains substituted with —OH or —SH. Preferably, Group VI includes serine, cysteine or threonine.

The term “deletion” as used herein includes exclusion of one or more amino acid residues (naturally occurring, non-naturally occurring, or peptidomimetic organic moiety) as compared to the original molecule from which it is derived.

The terms “insertion” or “addition” as used herein include the addition of one or more amino acid residues (naturally occurring, non-naturally occurring, or peptidomimetic (organic moiety) as compared to the original molecule from which it is derived.

The term “chemical modification” as used herein includes modification at the side chain of the amino acid residue, as well as modification of the peptidic bond. Accordingly, a functional group may be added to the side chain, deleted from the side chain or exchanged with another functional group. Typically, the modifications are conservative modifications resulting in conservative substitution. Examples of conservative modifications of this type include adding an amine or hydroxyl, carboxylic acid to the aliphatic side chain of valine, leucine or isoleucine, exchanging the carboxylic acid in the side chain of aspartic acid or glutamic acid with an amine or deleting the amine group in the side chain of lysine or ornithine. Other chemical modifications known in the art include arboxymethylation, acylation, phosphorylation, glycosylation or fatty acylation, and others.

The “chemical modification” also includes alteration of a bond within the peptidic backbone, i.e. that the bond between the N— of one amino acid residue to the C— of the next has been altered to non-naturally occurring bonds by reduction (to —CH₂—NH—), alkylation (methylation) on the nitrogen atom, or the bonds have been replaced by amidic bond, urea bonds, or sulfonamide bond, etheric bond (—CH₂—O—), thioetheric bond (—CH₂—S—), or to —C—S—NH—; The side chain of the residue may be shifted to the backbone nitrogen to obtain N-alkylated-Gly (a peptidoid). Modification also includes cyclization of the amino acid molecule, e.g. by forming S—S bonds. S—S bonds may be formed via the inclusion of sulphor-containing amino acid residues, such as cysteine at each terminus of the amino acid molecule. Cyclic peptides have been shown to be more stable and with higher biological activity than the corresponding linear molecule [Jining L. et al. Eur. J. biochem 271:2873-2886 (2004)].

Various fragments may be utilized in accordance with the present disclosure for raising there against antibodies. In the following examples, two synthetic peptides derived from PAR1-released peptide were selected and prepared (the N-terminal Cys residue being added for the purpose of antibody production).

Peptide PAR1rp1 having the sequence:
(SEQ ID NO. 2)
NH₂-C-S-A-R-T-R-A-R-R-P-E-S-K-A-COOH
Peptide PAR1rp2 having the sequence:
(SEQ ID NO. 3)
NH₂-R-R-L-L-L-V-A-A-C-F-S-L-C-G-P-L-L-S-A-R-COOH

It is noted that in the context of the present disclosure peptide PAR1rp2 is a preferred fragment of PAR1-released peptide for raising antibodies to be employed in the methods of the present invention.

The term “marker” is used herein to denote any component of a bodily fluid sample including any polymer, oligomer or small molecular weight compound. In one preferred embodiment, the marker is an amino acid-containing molecule, including a peptide, a polypeptide or a protein. In another embodiment, the marker comprises a PAR1-released peptide or a fragment thereof. The marker may comprise PAR1-released peptide or the fragment thereof in a free form or in the form of a complex with another one or more polypeptide, peptide or protein present in the fluid sample. In one embodiment, the marker is in the form of a complex with one or more blood proteins, as further mentioned below.

According to an embodiment, the fluid sample is a fluid from a body, selected from whole blood, plasma, serum, amniotic fluid, cerebrospinal fluid, ascitic fluid (ascites) or urine. Preferably, the bodily fluid sample comprises blood or blood serum.

The antibodies utilized by the methods disclosed herein are capable of binding to one or more PAR1-released peptides. The antibodies are generated by utilizing the PAR1-released peptide or the fragment derived from the PAR1-released peptide, as defined herein. The antibodies may be any one of polyclonal or monoclonal antibodies.

For the production of polyclonal antibodies, various hosts including goats, rabbits, rats, mice, etc. may be immunized by injection with PAR1-released peptide. Depending on the host species, various adjuvants may also be used to increase immunological response. Such adjuvants include but are not limited to Freund's, mineral gels such as aluminum hydroxide, and surface active substances such as lysolecithin, pluronic polyols, polyanions, peptides, oil emulsions, keyhole limpet hemocyanin, and dinitrophenol. BCG (bacilli Calmette-Guerin) and Corynebacterium parvum are potentially useful adjuvants.

Monoclonal antibodies may also be used. Monoclonal antibodies may be prepared using any technique which provides for the production of antibody molecules by continuous cell lines in culture. These include but are not limited to the hybridoma technique originally described by Koehler and Milstein (Nature 256:495-497, (1975)), the human B-cell hybridoma technique (Kosbor et al., Immunol. Today 4:72, (1983); Cote et al., Proc. Natl. Acad. Sci. 80:2026-2030, (1983)) and the EBV-hybridoma technique (Cole, et al., Mol. Cell Biol. 62:109-120, (1984); Chartrain M, Chu L. Development and production of commercial therapeutic monoclonal antibodies in Mammalian cell expression systems: an overview of the current upstream technologies. Curr Pharm Biotechnol. 2008;9(6):447-67; Price P W et al. Engineered cell surface expression of membrane immunoglobulin as a means to identify monoclonal antibody-secreting hybridomas. J Immunol Methods. 2009; Fransson J, Borrebaeck C A. Selection and characterization of antibodies from phage display libraries against internalizing membrane antigens. Methods Mol Biol. 2009; 480:113-27).

The term “cancer” in accordance with the invention shall mean any condition in which cells proliferate at an abnormally high and uncontrolled rate, the rate being more rapid than normal tissue growth. Generally, cancer may be broadly classified into three major types: Malignant tumors arising from epithelial structures (called carcinomas); malignant tumors that originate from connective tissues such as muscle, cartilage, fat or bone (called sarcomas); and malignant tumors affecting hematopoietic structures (structures pertaining to the formation of blood cells) including components of the immune system (called leukemias and lymphomas). Other neoplasms include but are not limited to neurofibromatosis.

The methods of the invention are of particular relevance to solid tumors. The term “solid tumor”, as is used herein, refers to any tumor which forms a mass. Tumor mass may show partial or total lack of structural organization and functional coordination with normal tissue and may be a primary tumor mass or a secondary tumor mass (i.e. as a result of cell migration from the original tumor site through the blood and lymph vessels). Examples of solid tumors include, but are not limited to, tumors of the brain, prostate, breast, colon, lung, kidney, bladder, liver, bone, head, neck, stomach, larynx, esophagus, ovary, cervix, hepatocarcinoma, lung carcinoma, rectum, colorectum and other sites in the gastrointestinal tract, uterus, ovary, skin (e.g., metastatic melanomas), endometrium, pancreas and testes.

It is noted PAR1 was found to be expressed in both primary and secondary breast cancer biopsys (data not shown). Thus, the present invention is applicable for determining both primary as well as secondary cancers.

In one embodiment, without being limited thereto, the cancer is a secondary cancer, i.e. a metastatic cancer.

In accordance with one preferred embodiment the cancer is primary or secondary breast cancer.

The methods disclosed herein are applicable for determining a cancer state, cancer severity as well as treatment efficiency at any stage of cancer as well as for determining recurrent cancer.

Overall, cancer stages may be referred to as Roman Numeral Staging. This system uses numerals I, II, III, and IV to describe the progression of cancer. Accordingly, cancer stages generally include:

Stage I: cancers localized to one part of the body; Stage II or III: cancers locally advanced (staging will depend on type of cancer); Stage IV: cancers which have metastasized, or spread to other organs or throughout the body.

The level of binding may be determined by quantitative as well as qualitative measuring. When referring to quantitative measurements, it may include determining the concentration of bound antibody, or of unbound antibody (determined e.g. by the use of a marked agent capable of binding to the free antibody). It is noted that this level correlates with the degree or severity of the disease. High level (above a predetermined threshold or in correlation with an a priori standard corresponding to a high cancer stage) is indicative of a sever state. With respect to the method for determining the effectiveness of treatment, and similarly, a decrease in the level is indicative in an improvement in the condition of a subject having cancer, e.g. as a result of an anti cancer treatment.

The terms “predetermined threshold” or “prior determined standards” are used herein to denote a reference value or range of values indicative of a healthy state or of a specific stage of a disease (degree of severity of the disease). When referring to a predetermined threshold (or prior determined standards) of a healthy state, the threshold (standards) may correlate with an averaged level of PAR1-released peptide from a statistically significant group of healthy subjects or a value or range of values otherwise derived from the level of PAR1-released peptide in a statistically significant group of healthy subjects. When referring to a predetermined threshold or prior determined standards of a specific stage of a disease, the threshold (standards) may be determined based on levels of PAR1-released peptide in a statistically significant group of patients diagnosed as having the disease in the specific stage of the disease. The manner of selecting the number of subjects used for a defining a group should be known to those versed in statistical analyses. Further, when referring to a predetermined threshold or prior determined standards of a specific stage of a disease, the threshold (standards) may be a reference point determined based on the level of PAR1-released peptide in a fluid sample taken from the examined subject at a time point, being when the subject was first diagnosed as having cancer or at a later time point. The level at that time point being the reference point for any follow up diagnosis of the subject's state making use of the method of the present invention.

Thus, in accordance with this embodiment, one or more first samples are taken at a time point prior to initiation of the therapeutic treatment (this being a reference point) and one or more second samples is taken at a time point during the treatment, wherein a decrease in the level of the binding exhibited in the at least one second sample as compared to that determined for the first sample is indicative that treatment is effective.

Alternatively, one or more first samples are taken at a time point during the treatment and one or more second samples are taken at a time point during the treatment subsequent to the time point of the one or more first samples, such that a decrease in the level of binding in the one or more second samples as compared to the one or more first samples is indicative that the treatment is effective.

Further, alternatively, one or more first samples are taken at a time point during the treatment and one or more second samples are taken at a time point after the treatment has been discontinued, wherein an increase in the level of binding in the one or more second samples as compared to the one or more first samples is indicative that the treatment is effective.

Many different detection systems are known in the art and can be utilized in the context of the present invention. These include competitive and non-competitive binding assays. Such systems include, without being limited thereto, techniques such as radioimmunoassays (RIA), enzyme immunoassays (EIA), enzyme linked immunosorbent assays (ELISA), “sandwich” immunoassays, immuno-precipitation reactions, gel diffusion reactions, immunodiffusion assays, agglutination assays, complement-fixation assays, immunoradiometric assays, fluorescent immunoassays, fluorescence polarization, protein A immunoassays, and immunoelectrophoresis assays. All these different detection system may be used for direct detection of the marker or by competition reactions.

In one embodiment, the detection is based upon a “sandwich” immunoassay, where the antibody, preferably, a monoclonal antibody is bound to a solid support. The fluid sample is then brought into contact with the solid support and any marker in the fluid sample is captured by the bound antibody. A second antibody which will bind to the marker can then be placed in contact with the solid support. The amount of marker in the sample can then be determined by detecting the amount of the bound second antibody.

The second antibody can be labeled, with, for example, a fluorescent compound, such as, without being limited thereto, fluorescein isothiocyanate, rhodamine, phycoerythrin, phycocyanin, allophycocyanin, o-phthaldehyde and fluorescamine; with a chemiluminescent compound, such as, without being limited thereto, luminol, isoluminol, theromatic acridinium ester, imidazole, acridinium salt and oxalate ester; a bioluminescent protein such as, without being limited thereto, luciferin, luciferase and aequorin; and radionuclides.

Also disclosed herein is diagnostic package for determining binding of an antibody raised against a protease-activated receptor 1 (PAR1)-released peptide or a fragment thereof to a marker within a fluid sample obtained from a subject, comprising:
(i) at least one antibody raised against a protease-activated receptor 1 (PAR1)-released peptide or a fragment thereof and capable of binding to said marker if present in said fluid sample;
(ii) instructions for use of said at least one antibodies for determining one or more of:
   binding of said antibody to said marker;
   level of binding of said antibody to said marker;
   difference between level of binding of said antibody to said marker and level of prior determined standards that correlate level of antibody binding to PAR1-released peptide with severity of cancerous state;
   difference in level of binding of at least one antibody to said marker in two or more successive fluid samples from same subject.
The package may comprise one or more of the following additional components:
   one or more additional antibodies capable of binding to said marker if present in the fluid sample, the one or more additional antibodies optionally being bound to a solid support, the one or more additional antibodies may be labeled, such as described above;
   instructions for use of said one or more additional antibodies.

The one or more antibodies may be capable of binding to PAR1-released peptide or fragment thereof. However, is some alternative embodiments, the one or more antibodies may be capable of binding to one or more peptides, polypeptides or proteins, other than PAR1, PAR1 released peptide or fragment thereof, that is present in the fluid sample in the form of a complex with PAR1, PAR1-released peptide or fragment thereof. This will allow the detection of PAR1, PAR1-released peptide or fragment in complex form. Examples of proteins, other than PAR1, PAR1 released peptide or fragment thereof which may form part of the complex in the context of the present invention include, without being limited thereto: Carboxypeptidase N and Complement Component 4 (C4).

The following non-limiting examples are provided for illustrative purposes only.

### NON-LIMITING EXAMPLES

### General

Two short synthetic peptides representing two (slightly overlapping) regions of PAR1 released peptide were prepared, PARr1p1 (SEQ ID NO:2) and PAR1rp2 (SEQ ID NO:3). Their sequences is shown based on the sequence of PAR1 released peptide, consists of 41 amino acids and having the following sequence

(SEQ ID NO: 1):

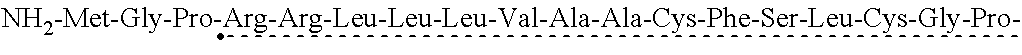

PAR1rp2 (SEQ ID NO: 3)

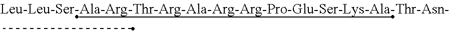

PAR1rp1 (SEQ ID NO: 2)
Ala-Thr-Leu-Asp-Pro-Arg-COOH

Polyclonal antibodies were raised against these two short synthetic peptides as described below.

Generally, the antibodies included:

A CUK-158 polyclonal antibodies raised against PAR1rp1, NH₂-C-S-A-R-T-R-A-R-R-P-E-S-K-A-COOH. SEQ ID NO:2;

Polyclonal antibodies raised against PAR1rp2, NH₂-R-R-L-L-L-V-A-A-C-F-S-L-C-G-P-L-L-S-A-R-CONH₂, SEQ ID NO:3, which were also used in the below exemplified immunodetection assay.

The antibodies raised against PAR1rp2 were immobilized on sepharose beads. The antibody-coupled beads were brought into contact with serum samples obtained from healthy or from patients diagnosed to have breast cancer (either before or after chemotherapeutic treatment, as discussed below). The beads containing serum samples were then subjected to an immunodetection assays.

### Materials and Methods:

### Peptide Synthesis and Polyclonal Antibody Production

The synthetic peptides and the polyclonal antibodies were produced by COVALAB S.A.R.L, FRANCE).

### Coupling of Antibodies to NHS-Activated Sepharose

Coupling buffer solutions were prepared containing 1 mM HCl, 0.1 M Tris pH 8, 0.1M acetate buffer 0.5M NaCl, pH 4.5, coupling buffer: 0.2M NaHCO₃, 0.1M NaCl pH 8.3); and then store at 4° C.

The antibodies were transferred into the coupling buffer solution and concentrated using amicon ultra-4 (Ultracel 30k with cutoff 30,000 MWCO). Neutralization of all Tris using Glycine, was verified (Tris may prevent binding to bead).

A step of washing the beads before adding the antibodies was conducted using 1 ml of NHS-activated sepharose (Amersham, Daniel Biotec representative) for 1.3 mg of antibodies (usually 0.5 ml). The NHS-activated sepharose were further washed with 10-15 ml (medium volumes) of cold 1 mM HCl immediately before use.

To the NHS-activated sepharose the antibodies were added (the ratio was 1 ml of NHS beads per 1.3 mg antibodies). The volume was then brought to 3 ml and the pH was adjusted to 6-8 (using the coupling buffer and 1 mM HCl)

The mixture was rotated at room temperature for 3 hours and then the beads were washed with 10-15 medium volumes of cold coupling buffer. In between washes the mixture was spindown for 1 min., at 1,200 rpm.

Blocking of the beads was achieved by adding 10-15 medium volumes of cold 0.1M Tris at pH 8, followed by rotation over night (minimum time for blocking −3 h) at 4° C.

The beads were then washed using a method that alternates high and low pH buffers. Specifically, the beads were washed with 10 ml of 0.1M Tris pH 8.0 and then with 10 ml of 0.1M acetate buffer 0.5M NaCl, pH 4.5. This washing was repeated 3 times (spin for 1 min at 1,200 rpm). The beads were then stored at 4° C. in 1× PBS with NaN₃.

### Antibody Biotinylation

As an alternative to coupling of the antibodies to sepharose beads, the antibodies may be biotinylated. To this end, anti-PAR1rp2 antibodies (1 mg are transferred into 0.1 M NaHCO₃ pH 8 buffer solution, and concentrated using Centricon (amicon ultra −4 Ultracel 30k with cutoff 30,000 MWCO). Tris is neutralized using glycine. Then a volume from the upper phase (in the Centricon ˜0.5 ml) is transferred to an eppendorf tube and diluted to 2 mg/ml with 0.1 M NaHCO₃ pH 8 (1 mg for example, should be in 0.5 ml). The antibodies are then transferred to a glass tube with a small magnetic stirrer. Biotin stock solution is prepared from 4.3 mg biotin in 100 μl DMF. Biotin (0.22 mg, Roche cat no. 11008960001) is added to every 1 mg antibody. The tube is then is covered with aluminum foil and is stirred for 3 hours at 4° C. and then 1 hour at room temperature.

The reaction is stopped with 1 μl of 1M NH₄Cl to each 100 μl of antibody-biotin solution (ratio 1 μl:100 μl) and stirred for 10 minutes at room temperature and spin down (in a plastic 50 ml tube 1 min, 1,200 rpm).

The resulting biotinilated antibodies are transferred to a centricon tube (amicon ultra −4 Ultracel 30k with cutoff 30,000 MWCO) and are washed once with PBS and then PBS (1× PBS solution) is added.

The resulting biotinilated is then diluted in glycerol 1:1 and store at −20° (2.8 mg/ml final 1.4 mg/ml in PBS and Glycerol). The biotinilated antibodies are divided to several tubes and stored.

### Blood Sample Preparation

Fresh blood sampled were extracted from patients. The samples were incubated in tubes at 37° C. for 10 minutes. Then, the samples were subjected to spinning at 3000 rpm for 10 minutes, 20° C. (room temperature, RT). From each tube, 1 ml serum samples in 1.5 ml micro tube with cap were put on ice and then frozen immediately at −80° C. It is further noted that aliquots of the serum were kept in 1 ml portions.

Immuoprecipitation of PAR1-Released Peptide from Serum with PAR1rp2 Antibodies Coupled Sepharose Beads

Frozen (−80° C. freezer) serum sample (1 ml) were mixed with 15 μl of protease inhibitor cocktail (Sigma). Protein A beads (100 μl) were then added (protein A beads may be replaced or combined with protein G beads) and the mixture was rotated for 1 hour at 4° C.

The serum samples/beads mixture were then spin down and transferred to a new eppendorf to which 500 μl 1× PBS and 80 μl of PAR1rp2 antibodies coupled to Sepharose beads (from 1:2 stoke in PBS; 40 μl packed+40 μl PBS) were added and the mixture further rotated for 2 hours at 4° C.

One hour after the beads were added, an additional serum sample was taken from the patient and the procedure above was repeated. The samples were then spin down.

The antibody bound beads were then washed 4 times with 800 μl of 50 mM Tris, 150 mM NaCl (pH 7.4).

Elution of the immunoprecipitated proteins was achieved by adding 50 μl of 0.1 M Glycine pH 2 to the beads and incubating the same for 10 minutes on ice and then spinning. The elute was transferred to a new eppendorf with Tris (10 μl, pH 8, to get the neutralizing pH).

Finally, 20 μl of non-reducing sample buffer and boiled for 5 minutes. The resulting samples were stored at −20° C.

Western Blot Analysis with Anti-PAR1rp2 Biotinylated Antibodies

First, blocking took place by adding the samples 3% BSA in TTBS, and shaking for 1 hour at RT.

To the samples anti-PAR1rp2 (the “first antibody”) in 3% BSA in TTBS (1:4000) was added 2.5 μg (microgram) antibodies. The mixture was shaken for 10 minutes at RT and incubated over night at 4° C. After overnight incubation, the samples were shaken again for 15 minutes.

The samples were then washed in TTBS 8×5 minutes.

To the samples a second antibody, streptavidin-HRP (Jackson, USA 2 μg) in 3% BSA in TTBS (1:10,000) was added and shaken for 30 minutes at RT. The samples were washed in TTBS 8×5 minutes.

The samples were detected using ECL (Pierce) reagent.

### Preparation of 10%-18% Gels

The gel comprises two gels, a lower (running) gel composed in the following manner: A lower running gel of 4 ml 18% polyacrylamide gel followed by another 4 ml of 10% polyacrylamide gel on top. The gels were solidified by adding ammnioum per sulfate (10% APS) and Temed. After the running gel were solid an upper layer of 3.5 ml upper gel was poured.

### Example 1

### Binding of Polyclonal Antibody to PAR1-Released Peptide

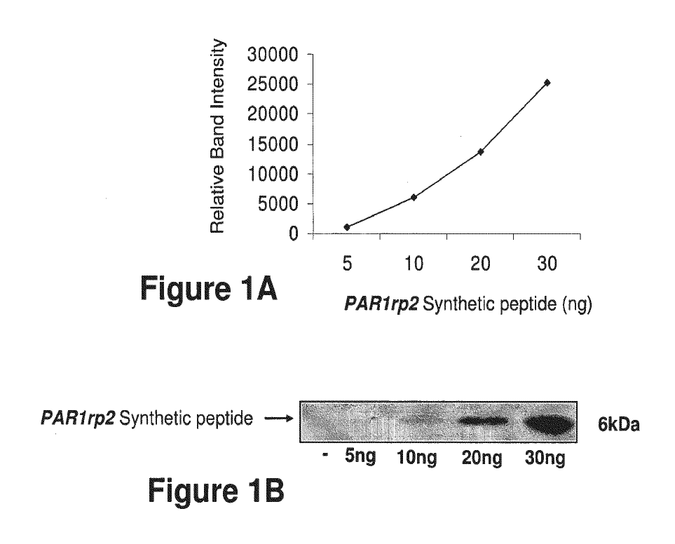

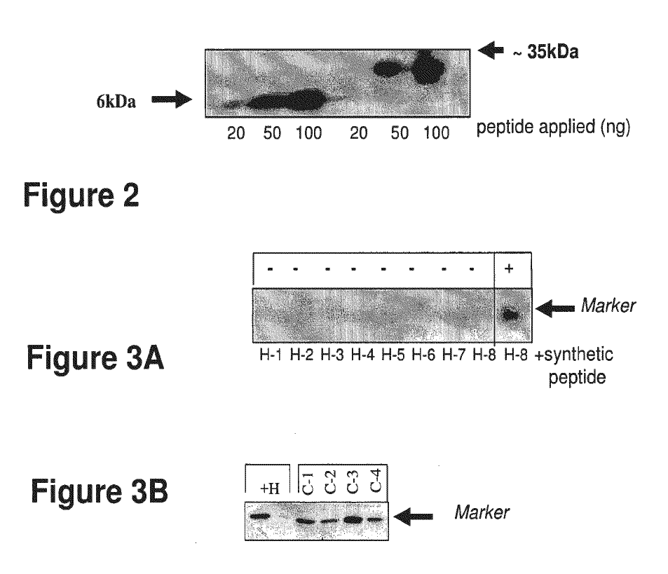

As a first feasibility test, the binding of the polyclonal antibody raised against PAR1rp2 to PAR1 released peptide was examined using Western Blot Analysis. FIGS. 1A and 1B show the binding of different amounts of the synthetic peptide PAR1rp2 (6 Ka in size) and FIG. 2 shows the binding of the polyclonal antibodies to the short synthetic peptide (PAR1rp2; 6 Ka) as well as to the intact peptide, namely, PAR1-released peptide (−35 kDa in size).

### Example 2

### Detection of Cancerous State Using Polyclonal PAR1rp2-Antibodies

PAR1rp2-antibodies were immobilized on the sepharose beads as described above. The antibody-coupled beads were brought into contact with serum samples obtained from healthy or from patients diagnosed to have breast cancer.

Each tested sample was then eluted from the bead with Glycine buffer (pH 2.0) neutralized by 0.1M Tris pH8.0. To the eluted material, sample buffer was added, boiled and separated on an SDS-PAGE gel. The gel (as described above) was blotted to a filter membrane and reacted with anti PAR1rp2 for detection. The gel was stained with comassie blue after blotting.

A total of 22 individuals that were a priori diagnosed as having breast cancer, and 24 individuals that were verified as non-cancer bearing (healthy) subjects, were examined. Using the detection method disclosed herein, the 22 patients were found to carry in their serum the cancer marker which was recognized by the polyclonal antibody raised against PAR1rp2. None of the healthy subjects were found to carry this marker.

In addition, western blot analysis was conducted for some randomly selected, albeit representatives, of the tested samples and the results are shown in FIGS. 3A-3B. Specifically, the presence of the marker was not detected in samples from healthy subjects (FIG. 3A). This was confirmed by spiking a serum sample from a healthy subject with the synthetic peptide PAR1rp2. On the other hand, the presence of the marker in the serum samples from cancer bearing patients was clearly determined (FIG. 313).

### Example 3

### Evaluation of Treatment Effectiveness

One aspect disclosed herein concern the use of polyclonal antibodies raised against a peptide derived from PAR1 released peptide for monitoring the effect of anti cancer treatment on cancer bearing subjects. It has been suggested that the PAR1 released peptide, detected in cancer serum patients, can serve as a reliable indicator for patient's response to a given anti-cancer treatment.

In the following example, blood was collected from two stage IV cancer patients (at two time points, a first time point before anti cancer treatment was given, to obtain a first serum sample, and a second time point being after 8 months of treatment with a cocktail of cyclophosphamide-methotrexate-5FFU, the other patient received hormonal treatment. To obtain a second serum sample. The level of binding of PAR1rp2 polyclonal antibodies to a marker in the first as well as in the second serum samples was determined. As control, at each time point two serum samples from a healthy subject were used, the first control was contacted with the polyclonal antibodies only (to confirm non-existence of the marker in the serum from healthy subjects) and the second control was contacted with a mixture of the polyclonal antibodies and the peptide PAR1rp2 (as proof that no marker indeed exist in the serum sample from the healthy subject and only by the external addition of the peptide, binding occurred).

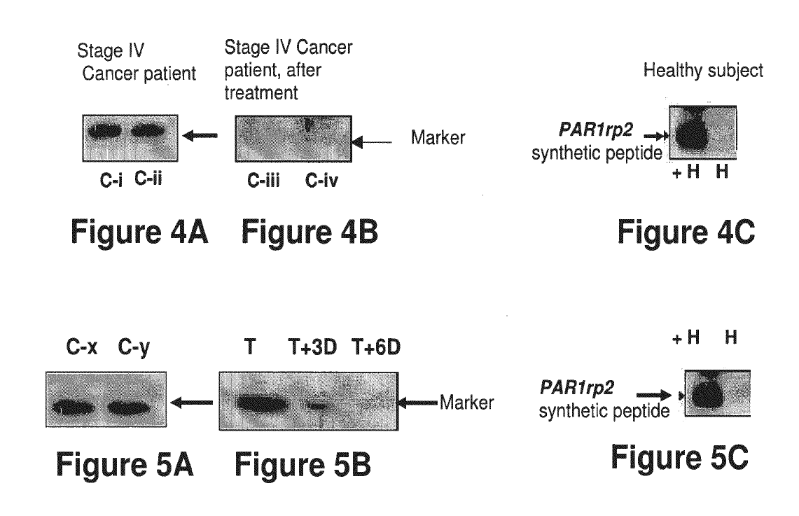

The results are presented in FIGS. 4A-4C. Specifically, FIG. 4A show that serum samples from the cancer patients prior to treatment contained the marker (FIG. 4A), while after treatment the level of the marker was significantly reduced (FIG. 4B). With respect to serum samples from a healthy subject (the control, FIG. 4C), binding was detected only when the synthetic peptide was introduced. It is noted that the result correlated with conventional methods for determining effectiveness of treatment including CT scan and levels of the marker CEA 15-3.

Thus, the method disclosed herein may also be used for individual patient follow-up at times intervals a mean of disease progression reporter. To this end, once a subject is diagnosed with cancer, the level of the marker may be measured and this level will be used as a reference point for any follow-up analysis of the subjects cancerous state.

To verify that no false results may occur using the method according to the invention due to, for example, existing inflammation or as a result of trauma, the level of PAR1 released peptide in subjects under trauma was determined. It was hypothesized that in trauma patients, even if the level of the peptide increases after injury, it will reduce once the trauma is ameliorated. The same would occur with inflammation. To prove this hypothesis, first blood samples from trauma patients (e.g. after surgery or wounded) were collected on the day of trauma and the level of PAR1 released peptide in the serum was determined using polyclonal antibodies raised against PAR1rp2. Second blood samples were collected from the same patients, several days after the trauma was treated. FIGS. 5A-5C show the level of the marker in the blood samples from a cancer patient (“C”, FIG. 5A), from day 1 trauma patient (“T”, FIG. 5B), from the same trauma patient 3 days after abdominal trauma (“3D” FIG. 5B) and then after an additional 3 day period (total of 6 days after injury “6D” FIG. 5B). As control, level of the marker in a blood sample from a healthy patient was used without or with the synthetic peptide (“H”, “+H”, FIG. 5C). The results show that while in the day of the injury, high level of PAR1 released peptide was observed (similar to the level in cancer patient), 3 days after the injury and even more 6 days after injury, the level of the marker was dramatically decreased.

Without being bound by theory, it is expected that patients having an inflammatory condition will exhibit a similar peptide level profile, i.e. during the inflammatory state, the level will be high, however, once the inflammation is treated or relieved, the level of the peptide will dramatically decrease.

### Example 4

### Determining Disease Stage

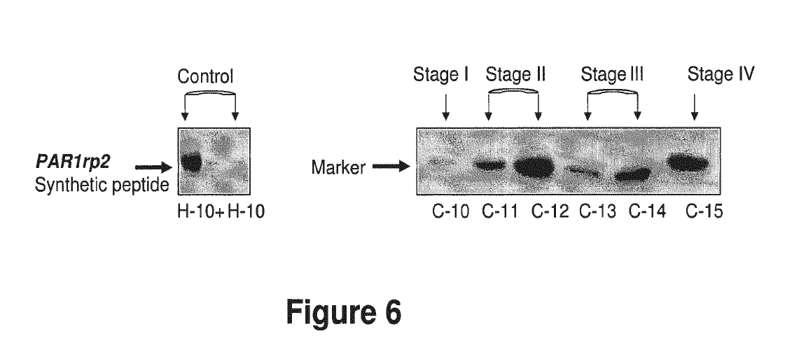

A further aspect disclosed herein concerns determining of a cancerous stage in a subject diagnosed with cancer. It was found that the marker detected in all cancer stages and that the level of the marker may be used to profile the stage of cancer. Specifically, FIG. 6 shows that the marker is detected at all cancer stages and at different levels. This may allow the early detection of cancer (even at stage I or II) as well as the profiling of each stage, and the characterizing a stage according to its aggressiveness or potential aggressiveness. To this end, it is noted that the level of the binding at each stage may vary depending on the aggressiveness of the stage. Thus, for example, a high level of the marker (i.e. statistically significantly higher than the predetermined threshold/reference point) at stages I and/or II of the disease may be indicative of an aggressive cancer.

The invention has been described in an illustrative manner, and it is to be understood that the terminology which has been used, is intended to be in the nature of words of description rather than of limitation. Obviously, many modifications and variations of the present invention are possible in light of the above teaching. It is therefore, to be understood that within the scope of the appended claims, the invention may be practiced otherwise than as specifically described hereinafter.

In this connection, it is noted that as used in the above description and following claims, the forms “a”, “an” and “the” include singular as well as plural references unless the context clearly dictates otherwise. For example, the term “an antibody” includes one or more plurality of antibodies.

Further, as used herein, the term “comprising” is intended to mean that the methods or packages includes the recited elements, but not excluding others. Similarly, “consisting essentially of” is used to define methods or packages that include the recited elements but exclude other elements that may have an essential significance on the performance of the methods disclosed herein. “Consisting of” shall mean excluding more than trace elements of other elements. Embodiments defined by each of these transition terms are within the scope of this invention.

Further, all numerical values, e.g., concentration or dose or ranges thereof, are approximations which are varied (+) or (−) by up to 20%, at times by up to 10% of from the stated values. It is to be understood, even if not always explicitly stated that all numerical designations are preceded by the term “about”. It also is to be understood, although not always explicitly stated, that the elements described herein are merely exemplary and that equivalents of such are known in the art.

1. A method of determining a cancerous state in a subject, the method comprising determining binding of an antibody raised against a protease-activated receptor 1 (PAR1)-released peptide or a fragment derived therefrom to a marker within a fluid sample obtained from said subject, wherein binding of said antibody to said marker is indicative of a cancerous state.

2. The method of claim 1 further comprising determining severity of a cancerous state in a subject comprising determining level of binding of an antibody raised against PAR1-released peptide or a fragment derived therefrom to a marker within a fluid sample obtained from said subject, and comparing the level of binding with the level of prior determined standards that correlate level of antibody binding to PAR1 released peptide with severity of cancerous state.

3. A method determining the effectiveness of a therapeutic treatment of a subject with an anti-cancer agent comprising determining the level of binding of an antibody raised against PAR1-released peptide or a fragment derived therefrom to a marker within a fluid sample obtained from said subject at two or more successive time points, one or more time points being during the therapeutic treatment, wherein a difference in the level is indicative of effectiveness of therapeutic treatment.

4. The method of claim 1, wherein said PAR1-released peptide comprises an amino acid sequence as depicted in SEQ NO:1.

5. The method of claim 4, wherein said PAR1-released peptide consists of an amino acid sequence as depicted in SEQ ID NO:1.

6. The method of claim 1, wherein said fragment of PAR1-released peptide comprises at least 5 consecutive amino acid residues corresponding to consecutive amino acid residues in said PAR1-released peptide when the fragment and the PAR1-released peptide are optimally aligned and wherein said at least 5 consecutive amino acid residues are identical to 5 consecutive amino acid residues of PAR1-released peptide.

7. (canceled)

8. The method of claim 1, wherein said fragment comprises a conservative modification of one or more amino acid residue in said PAR1-released peptide, said modification is selected from insertion of an amino acid, deletion of an amino acid, substitution of an amino acid, chemical modification of an amino acid.

9. The method of claim 8, wherein said modification comprises insertion or substitution with a different amino acid residue selected from a naturally occurring amino acid, a non naturally occurring amino acid or a peptidomimetic residue.

10. The method of claim 1, wherein said fragment comprises the sequence as depicted in SEQ ID NO:2 or SEQ ID NO:3.

11. The method of claim 10, wherein said fragment comprises the sequence depicted in SEQ ID NO:3.

12. (canceled)

13. (canceled)

14. The method of claim 13, wherein said marker comprises a PAR1 -released peptide or a complex of said PAR1-released peptide with at least one other component present in said fluid sample wherein said another component is a protein, polypeptide or peptide present in said fluid sample.

15. (canceled)

16. (canceled)

17. The method of claim 16, wherein said antibody is a polyclonal antibody raised against a peptide having the sequence depicted in SEQ ID NO: 3.

18. The method of claim 1, wherein said fluid sample is selected from the group consisting of whole blood, plasma, serum, amniotic fluid, cerebrospinal fluid, ascitic fluid and urine.

19. (canceled)

20. The method of claim 2, wherein determination of the level of binding of said antibody to said marker comprises a quantitative measurement of said level, a qualitative measurement of said level or combination of same.

21. The method of claim 2, wherein said prior determined standards are determined by measuring levels of binding of said antibody to the marker in fluid samples obtained from a statistically significant group of cancer patients having defined severities of cancer or by measuring level of binding of said antibody to the marker in fluid sample obtained from subject at a prior reference time point.

22. The method of claim 2, wherein said comparing of the level of binding with the level of prior determined standards comprises qualitative comparison, quantitative comparison or combination of same.

23. The method of claim 3, wherein least one first fluid sample is taken at a time point prior to initiation of the therapeutic treatment and at least one successive sample is taken at a time point during the treatment, wherein a decrease in the level of the binding exhibited in the at least one successive sample as compared to that determined for the first fluid sample is indicative that treatment is effective.

24. The method of claim 3, wherein at least one first fluid sample is taken at a time point during the therapeutic treatment and at least one successive fluid sample is taken at a time point during the therapeutic treatment subsequent to the time point of the first sample, such that a decrease in the level of binding in the successive sample as compared to the level in the first sample is indicative that the treatment is effective.

25. The method of claim 3, wherein at least one first fluid sample is taken at a time point during the therapeutic treatment and at least one successive fluid sample is taken at a time point after the treatment has been discontinued, wherein an increase in the level of binding in the one or more second samples as compared to the one or more first samples is indicative that the treatment is effective.

26. A package for determining binding of an antibody raised against a protease-activated receptor 1 (PAR1)-released peptide or a fragment thereof to a marker within a fluid sample obtained from a subject, comprising:
(i) at least one antibody raised against protease-activated receptor 1 (PAR1)-released peptide or a fragment thereof and capable of binding to said marker if present in said fluid sample: and
(ii) instructions for use of said at least one antibody for determining one or more of:
(a) binding of said antibody to said marker;
(b) level of binding of said antibody to said marker;
(c) difference between level of binding of said antibody to said marker and level of prior determined standards that correlate level of antibody binding to PAR1-released peptide with severity of cancerous state; or
(d) difference in level of binding of at least one antibody to said marker in two or more successive fluid samples from the same subject.

27. (canceled)

28. (canceled)
